(19) United States
(12) Patent Application Publication
(10) Pub. No.: US 2011/0262612 A1
(43) Pub. Date: Oct. 27, 2011

(54) ROSEHIP POWDER HAVING SMALL PARTICLE SIZE

(76) Inventors: Nicolle Götz, Waldshut-Tiengen (DE); Ullrich Kranzer, Munchen (DE); Swen Wolfram, Waldshut-Tiengen (DE)

(21) Appl. No.: 12/988,143

(22) PCT Filed: Apr. 17, 2009
(86) PCT No.: PCT/EP2009/054584

§ 371 Date: Dec. 28, 2010

(30) Foreign Application Priority Data
Apr. 17, 2008 (EP) 08007493.3

(51) Int. Cl. A23L 1/30; A23C 9/13; A23G 3/48; A23K 1/14; A21D 13/08; A23L 2/52; A23L 1/212

(52) U.S. Cl. 426/583; 426/640; 426/635; 426/620; 426/590; 426/519; 426/648

(57) ABSTRACT

Rosehip powder has been finely ground such that it contains essentially no particles larger than 600 um when measured by laser diffraction. In one aspect of the invention (claim 1) 50% of the particles have a size of 100-150 microns. The fine powder can be used in foodstuffs which are subsequently high pressure homogenized, such as smoothies and milk based beverages. Additionally, the fine powder can be added to yoghurts and other juices while retaining acceptable sensory characteristics.

### FIELD OF THE INVENTION

This invention relates to fine rosehip powders which have been processed so that substantially all the particles are smaller than 600 microns when measured using a laser diffraction method, and preferably at least 80% of the powder by weight would pass through a sieve with openings of 300-425 microns, and at least 60% by weight would pass through a sieve with openings of 150-200 microns. This invention also relates to foods and cosmetics containing the fine particle sized rosehip powder.

### BACKGROUND OF THE INVENTION

Rosehips from plants such as Rosa canina are high in vitamins, and can be used for the treatment and prophylaxis of arthritis and other inflammatory conditions. They have a tangy, yet sweet flavor, and have historically been used to make tea, perserves (including jam, jelly and marmelades) soups (such as nyponsuppa in Sweden), syrups, cordials, and the like. Alternatively, rosehip powder can be consumed in the form of a capsule, such as that sold under the trademark“i-FLEX”.

U.S. Pat. No. 6,024,960 to Kharazmi et al discloses a rosehip powder made by harvesting rose hips, chopping them into pieces, optionally freezing them, and then drying the rose hips to reduce water content to about 5% by weight. The dried chopped rose hips are then passed through a separator to remove nuts, hairs and other unwanted matter. The remaining fruit is then crushed into a powder in a grinding mill. The specification teaches that the so-formed powder has a particle size of below 1 mm, with about 0.1 to 0.5 mm being preferred. This powder is commercially available under the trademark HYBEN-VITAL rose hip powder, and also under the trademark i-FLEX sold by DSM.

U.S. Patent application publication No. US 2007/0184164 discloses a food premix which contains a combination of powdered fruit seed or fruit seed fraction which has a particle size of 20 mesh or finer (841 microns). The powder is blended with a dry food material to form a premix; and the premix is incorporated into a food product which is supplemented with a-linolenic acid, dietary fibers, antioxidants phytochemicals or a combination thereof.

Spiro et al 1993 Food Chemistry 48:39-45 describes rose hip teas made by cutting whole rose hips in half, removing the seeds and hair, and then grinding in an electrical coffee grinder to size fractions of 424-500 microns, 600-710 microns, 850-1000 microns, and 1180-1400 microns.

Chinese Patent 101040833 (Tianjin Yumelijing Group Co., Ltd) discloses a cosmetic product which contains a variety of ingredients, including rose hip pulp which has a size of 5-200 mesh. (200 mesh is the equivalent of 90 microns; 5 mesh is 4000 microns).

Japanese Kokai 2004275015 2004 (K. Emoto) discloses use of rose hip powder with an average size of ≦2 mm as a gelling agent for the manufacture of fruit jams.

Soviet Union Patent 1002350, 1983, (Kharkov Scientific-Research Chemical-Pharmaceutical Institute, USSR) discloses a process for increasing the yield of rose hip oil from seeds by grinding seeds to 0.05-3 mm and then extracting with dichlorodifluoromethane.

Certain physical properties of commercially available rosehip powder prevents its use in a larger variety of consumer foods. It would be desirable to have rosehip powder in a form which allows more consumers to benefit from its high nutritional characteristics.

### BRIEF DESCRIPTION OF THE INVENTION

In accordance with this invention, it has been found that rosehip powder which has a defined size range has physical characteristics which improve its processing and sensory characteristics, and thus can be added to many new foodstuffs and even cosmetic preparations.

Thus one aspect of this invention is a finely ground rosehip powder which has substantially no particles larger than 600 microns when measured by laser diffraction. Another aspect of this invention is a population of rosehip powder particles where 50% of the particles (by volume) have a size of 125 microns (plus or minus 25 microns), when measured by Malvern Laser Diffraction Method.

Another aspect of this invention is a finely ground rosehip powder which has particle sizes such that 80% would pass through a sieve which has openings of 300-424 microns and at least 60% by weight would pass through a sieve which has openings of 150-200 microns.

Another aspect of this invention is a finely ground rosehip powder in which over 80% by weight of the particles are smaller than 425 microns, and which at least 70% by weight are smaller than 300 microns when measured using a sieve.

As used throughout the claims and specification, the following definitions apply. “Coarse” powder refers to rosehip powders which have particle sizes which equal or exceed 700 microns (um) when measured using the laser diffraction method.

“Medium coarse” powder refers to powder which is has a particle size which is smaller than the coarse powder, but has a fraction which is larger than 600 microns. Its average particle size is approximately 400 microns.

“Fine” powder refers to rosehip powder according to this invention which has substantially no particles larger than 600 microns when measured using the laser diffraction method. Further, at least 80% of the powder (by weight) would pass through a sieve whose openings are 300-425 microns, and at least 60% (by weight) would pass through a sieve whose openings are 150-200 microns.

“Substantially no particles larger than 600 microns” means that at least 95%, more preferably at least 98% of all particles in the powder are smaller than 600 microns when measured using a laser diffraction method.

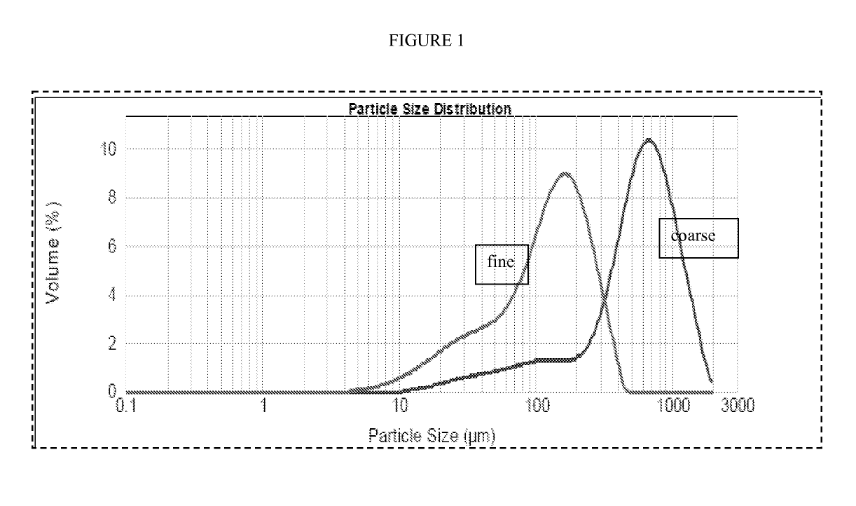

FIG. 1 is a Particle Size Distribution graph showing populations of coarse and fine rosehip powders measured using the laser diffraction method. “Coarse” refers to commercially available HYBEN VITAL powder. “Fine” refers to a powder which has substantially no particles larger than 600 microns.

The powders of this invention may be from any plant which is recognized as a source of rose hips suitable for human consumption. This would include Rosa canina, Rosa gallica, Rosa condita, and Rosa rugosa. Rosa canina (dog rose) is a preferred plant source of the rose hips. The rose hip powders of this invention may include the entire rose hip, or it may include only selected parts. In a preferred embodiment, only the shells are used.

The description of particle size of powders is a complicated task. There are variety of ways to measure particle size, and the information obtained from each method is not interchangeable with data obtained from using a second method. Thus, when measuring particle size, particularly for irregularly shaped particles (such as rosehip powder), is it necessary to specify which method of measurement has been used.

One standard, and fairly simple means of measurement is to pass the powder through a series of graded sieves. Each sieve has a smaller opening in the mesh than the sieve above it, and will trap particles larger than the opening. When using this means for measuring the fine powder of this invention, approximately 97.6% of the particles had a size which was less than 600 microns. Approximately 93% of the particles had a size which were smaller than 425 microns, and approximately 60% had a size which was smaller than 200 microns. Further details of this particle population is given in EXAMPLE 1. Thus one aspect of this invention is a rosehip powder which, when measured using a sieve method, has a population which substantially no particles are larger than 600 microns, and at least 60% (by weight) of the particles are smaller than 200 microns.

A second type of particle size measurement is performed using the laser diffraction method. This test measures the light scattering pattern of the particles which are suspended in a laser beam, and calculates the diameter of a sphere which would yield an equivalent light scattering pattern. To a good approximation, this corresponds to the sphere of equivalent average cross-sectional area. The laser system can then calculate the volume percentages of particles that have a given size. This corresponds to a mass distribution (assuming that particle density is the same for all sizes). A comparison of the particle size distribution between commercially available rosehip powder and the rosehip powder of this invention as determined by laser diffraction can be seen in FIG. 1. Thus, another aspect of this invention is a rosehip powder which, when measured using a laser diffraction method, has a population which substantially has no particles which are larger than 600 microns, and particles of 125 microns (plus or minus 25 microns) account for 50% of the volume of the population.

Currently available rosehip powder cannot be incorporated as an ingredient in many processed foods, such as yoghurts, smoothies, and juices. Aside from the graininess which imparts an unpleasant feeling in the mouth, the powder can separate or segregate or lack solubility or dispersability. For example, with many beverages, the larger particles fall out of solution rapidly, leaving a “mud” or sediment at the bottom of the container. Also, the larger size particles impart a viscosity to the liquid which makes high pressure homogenization impossible.

Thus, one aspect of this invention is a water based beverage containing fine rosehip powder. The fine rosehip powder, either alone or in combination with other powdered beverage forms, can be added by the consumer to water (or other water based drinks), shaken or stirred, and then consumed. The fine powder disperses better than the normal powder and will stay in solution better. Further it has a less grainy mouthfeel than the larger sized powders.

It has been found, in accordance with this invention, that the fine particle size powder can be used to make drinks and other foodstuffs which are subsequently subjected to high pressure homogenization. Thus one aspect of this invention is a foodstuff which contains rosehip powder and which has been high pressure homogenized, such as a rose-hip powder containing smoothie, milk based drinks such as milkshakes, yoghurt, or fruit juice. Another aspect of this invention is a method of preparing a liquid food product comprising adding fine rosehip power to a liquid food to obtain a rosehip powder containing food, and homogenizing the rosehip powder containing liquid food under high pressure.

Thus another aspect of this invention are foodstuffs containing rosehip powder which has substantially no particles which are larger than 600 microns when measured using laser diffraction, with the proviso that the foodstuff is not tea.

The foodstuffs of this invention may be beverages or solid foods. Food, as used throughout this specification and claims, can encompass both food for humans and food for animals (including canine, feline, and farm animal feeds, and premixes).

Examples of beverages according to this invention include, but are not limited to: soft drinks, water based drinks, yoghurt drinks, smoothies, other milk based drinks, including milkshakes, soy-based drinks, and juices. Examples of foodstuffs which are not particularly preferred are teas, soups, or infusions where the ingredients of the powder are extracted using water or other solvent, and then consumed, as particle size has no particular benefit.

Examples of food where the rosehip powders of this invention can be utilized include: cereals, cereal bars, cookies, cakes and other baked goods, confectionery, dairy based foods, soups and sauces, dressings, marmalades, ice cream, puddings, chewable candies, chews, lozenges, gummi bears (and other gummi sweets), and chewing gums. Candies, such as chocolates (dark chocolate, milk chocolate, and white chocolates are particularly preferred), and may be consumed on their own or may be used as frostings, fillings, or topping for cakes, cereal bars or other baked goods.

Additionally, the rosehip powder of this invention may be formulated into capsules or tablets as a nutraceutical.

A “smoothie” is a blended, usually chilled, sweet beverage made from fresh fruit. It is sometimes blended with crushed ice, frozen fruit, or frozen yogurt. Smoothies have a milkshake-like consistency which is thicker than slush drinks, but unlike milkshakes, they do not usually contain cow's milk or ice cream. Smoothies are marketed to health-conscious people, and some restaurants offer add-ins such as soy milk, whey powder, green tea, herbal supplements, or nutritional supplement mixes.

Rosehip powder has been added to a fruit smoothie before production to test the possibility of high pressure homogenisation and sensory acceptability of different levels in one serving (100 ml). The coarse powder and medium-coarse powder were not suitable as both forms could not be high pressure homogenised (too coarse). With the fine powder material we were able to produce a smoothie containing up to 5 g rosehip powder/100 ml.

The following Examples are presented to better illustrate the invention.

### EXAMPLES

### Example 1

### Preparation of Fine Rosehip Powder

### Sieve Analysis

Commercially available HYBEN-VITAL rose hip powder is sized using the sieve method. Results are presented in Table 1A, below.

TABLE 1A
SIZE OF COARSE ROSEHIP POWDER

| | | weight | weight sieve | weight | | weight | weight |
|---|---|---|---|---|---|---|---|
| Mesh | | empty | with powder | powder | | powder | through |
| [μm] | | sieve [g] | [g] | [g] | | [%] | sieve [%] |
| < | 150 | 328.0 g | 329.9 g | 1.9 | g | 1.2% | 1.2% |
| 150 | 200 | 259.5 g | 320.8 g | 61.3 | g | 40.2% | 41.4% |
| 200 | 250 | 275.0 g | 289.1 g | 14.1 | g | 9.2% | 50.7% |
| 250 | 300 | 280.8 g | 285.9 g | 5.1 | g | 3.3% | 54.0% |
| 300 | 425 | 280.7 g | 294.4 g | 13.7 | g | 9.0% | 63.0% |
| 425 | 600 | 285.1 g | 309.2 g | 24.1 | g | 15.8% | 78.8% |
| 600 | 800 | 315.8 g | 346.2 g | 30.4 | g | 19.9% | 98.7% |
| 800 | 1000 | 319.1 g | 320.8 g | 1.7 | g | 1.1% | 99.8% |
| > | 1000 | 331.4 g | 331.7 g | 0.3 | g | 0.2% | 100.0% |
| | | | sum | 152.6 | g | 100.0% | |

Rosehip powder is milled according to standard methods, and is sized using a series of sieves. Results are presented below.

TABLE 1B
SIZE OF FINE POWDER PARTICLES

| | | weight | weight sieve | weight | | weight | weight |
|---|---|---|---|---|---|---|---|
| SIZE | | empty | with powder | powder | | powder | through |
| um | | sieve [g] | [g] | [g] | | [%] | sieve [%] |
| < | 150 | 328.0 g | 362.4 g | 34.4 | g | 34.5% | 34.5% |
| 150 | 200 | 259.5 g | 286.7 g | 27.2 | g | 27.3% | 61.8% |
| 200 | 250 | 275.0 g | 282.8 g | 7.8 | g | 7.8% | 69.6% |
| 250 | 300 | 280.8 g | 283.1 g | 2.3 | g | 2.3% | 71.9% |
| 300 | 425 | 280.7 g | 292.0 g | 11.3 | g | 11.3% | 83.2% |
| 425 | 600 | 285.1 g | 294.8 g | 9.7 | g | 9.7% | 93.0% |
| 600 | 800 | 315.4 g | 320.0 g | 4.6 | g | 4.6% | 97.6% |
| 800 | 1000 | 319.2 g | 321.0 g | 1.8 | g | 1.8% | 99.4% |
| > | 1000 | 331.5 g | 332.1 g | 0.6 | g | 0.6% | 100.0% |
| | | | sum | 99.7 | g | 100.0% | |

As can be seen, 97.6% of the powder is smaller than 800 um; 93% is smaller than 600 um; and 83.2% is smaller than 425 um.

### Example 2

### Malvern Laser Diffraction

Two rosehip powders, the commercially available HYBEN VITAL powder and powder which had been further milled (Fine Powder) were analyzed using conventional laser diffraction analysis methods. This method measures the volume of particles which have a given particle size. Results are presented in FIG. 1.

Additionally, the following particle sizes were calculated:

TABLE 2
Particle sizes

| Product: | d(0.1) | d(0.5) | d(0.9) |
|---|---|---|---|
| Rosehips Standard | 114.315 μm | 590.199 μm | 1163.279 μm |
| Rosehips Fine Powder | 27.564 μm | 125.593 μm | 265.189 μm |

d(0.1) refers to the size of the particles which make up the smallest 10% volume of the population. d(0.5) refers to the size of the particles which make up 50% volume of the population; and d(0.9) refers to the volume of the particles making up 90% of the population. As can be seen from FIG. 1, The Fine Powder had substantially no particles which were bigger than 600 microns, and substantially no particles which were bigger than 500 microns. In contrast, the average particle size of the commercially available powder was approximately 600 microns.

### Example 3

### Use in Foodstuffs

Medium Coarse rose hip powder was made by grinding coarse rosehip powder in a conventional mill until particle size averaged approximately 400 um using the laser diffraction method.

TABLE 3
Material used

| Product name | LOT | Remarks |
|---|---|---|
| Rosehip powder | Retail | Hyben Vital, ~700 μm particle size; by |
| “Coarse” | packaging | laser diffraction |
| Rosehip powder | V168A* | ~400 μm particle size by laser |
| “Medium coarse” | | diffraction |
| Rosehip powder | V168B* | ~200 μm particle size by laser |
| “Fine” | | diffraction |

*delivered by Vital Products GmbH, MAX.FOOD GmbH, 98631 Wolfmannshausen, Germany

All samples of foods were given to a trained taste panel. The sensory analysis was performed by means of descriptive analysis by using interval scales in terms of different attributes. The interval scale consists of 7 intervals, starting with 1 for attribute not detectable up to 7 for extremely intense. An analysis of variance (ANOVA) was carried out to see if there was a significant difference. Multiple comparisons were made with the least significant difference test (L.S.D.) at 5% level of significance.

In a second test the panel could choose between samples to indicate preferences for different milling grades of the rosehip powders.

All samples were stored in the refrigerator. Results are given in the following examples.

### Example 4

### Yogurts

Fine rosehip powder or a coarse rosehip powder were simply added (mixed in) to plain flavored commercially available yoghurt, to judge their sensory advantages or disadvantages. Results are presented below.

TABLE 4
Sensory profile of rosehip powder in yoghurt (simply mixed in)

| | | L.S.D. | | L.S.D. | | L.S.D. | | L.S.D. | | L.S.D. | Off- | L.S.D. | | L.S.D. |
|---|---|---|---|---|---|---|---|---|---|---|---|---|---|---|
| | sweet | 5% | sour | 5% | bitter | 5% | fruity | 5% | grainy | 5% | taste | 5% | color | 5%% |
| Control | 1.82 | — | 4.09 | — | 1.36 | B | 1.09 | B | 1.00 | C | 1.55 | — | 1.09 | B |
| 200 μm | 1.82 | — | 4.18 | — | 1.91 | A | 2.00 | A | 2.82 | B | 2.36 | — | 4.91 | A |
| 700 μm | 1.82 | — | 4.45 | — | 1.73 | AB | 1.55 | AB | 5.64 | A | 2.00 | — | 4.09 | A |
| ANOVA | not sign. | | not sign. | | sign. | | sign. | | sign. | | not sign. | | sign. | |
| L.S.D. diff. | | — | | — | | 0.41 | | 0.7 | | 0.71 | | — | | 0.85 |

5 g powder used in 180 g material

Rosehip powder changed the sensory profile of plain yoghurt (no other flavor added) significantly. The yoghurt taste became fruitier, more grainy and showed an intensive orange colour. While it also became slightly more bitter, this was not judged to be an unacceptable characteristic. The finer material was judged as more pleasant by ˜67% of the panelists as it is not as grainy as the coarser material.

### Yogurt Processing

Rosehip powder was added to milk before producing a yoghurt to test the possibility of high pressure homogenization and sensory acceptability of different levels of powder. The coarse and medium coarse powders were found not to be suitable as both forms could not be high pressure homogenized. In contrast, with the fine material, we were able to produce a yoghurt containing 3.5 g rosehip powder per serving of 150 g.

Rosehip powder at a level of 3.5 g/150 g yoghurt changed the sensory profile regarding graininess and sourness. Some panelists detected an off-taste, mostly described as “earthy”. Differences could be detected in plain flavored yoghurt but are most likely not that obvious in fruit flavored yoghurt.

TABLE 10
Sensory of rosehip powder in a yoghurt (processed with rosehips powder)

| | | L.S.D. | | L.S.D. | | L.S.D. | | L.S.D. | Off- | L.S.D. |
|---|---|---|---|---|---|---|---|---|---|---|
| | sweet | 5% | sour | 5% | fruity | 5% | grainy | 5% | taste | 5% |
| Control | 2.25 | — | 3.92 | B | 1.33 | — | 1.00 | B | 1.00 | — |
| 3.5 g/150 g | 2.58 | — | 2.75 | A | 1.92 | — | 2.83 | A | 1.33 | — |
| ANOVA | not sign. | | sign. | | not sign. | | sign. | | not sign. | |
| L.S.D. diff. | | — | | 0.53 | | — | | 0.85 | | — |

### Example 5

### Orange Flavored Instant Drink

TABLE 5
Sensory profile of rosehip powder in an orange flavoured instant drink

| | | L.S.D. | | L.S.D. | | L.S.D. | | L.S.D. | | L.S.D. | Off- | L.S.D. | | L.S.D. |
|---|---|---|---|---|---|---|---|---|---|---|---|---|---|---|
| | sweet | 5% | sour | 5% | bitter | 5% | fruity | 5% | grainy | 5% | taste | 5% | color | 5%% |
| Control | 3.58 | — | 4.42 | — | 1.75 | — | 1.00 | B | 1.00 | B | 1.08 | — | 3.00 | — |
| 200 μm | 3.67 | — | 4.00 | — | 1.50 | — | 2.92 | A | 3.17 | A | 1.67 | — | 4.67 | — |
| 700 μm | 3.42 | — | 4.25 | — | 1.67 | — | 2.17 | A | 3.92 | A | 1.33 | — | 3.75 | — |
| ANOVA | not | | Not | | Not | | Sign. | | Sign. | | Not | | Not | |
| | sign. | | sign. | | sign. | | | | | | sign. | | sign. | |
| L.S.D. | | — | | — | | — | | 0.85 | | 0.97 | | — | | — |
| diff. | | | | | | | | | | | | | | |

Rosehip powder changed the sensory profile of an orange flavored instant drink significantly. The drink became fruitier, more grainy and showed an intensive orange colour. The difference between the attribute ‘grainy’ is not significant between the 200 and 700 μm material, most likely because the larger particles sedimented to the bottom of the beaker and not the whole sample was not consumed. The finer material colored the drink more homogeneously.

### Example 6

### Smoothie

Various smoothies containing differing amounts of rosehip powders were prepared according to the following:

TABLE 6
Smoothie recipies

| | Sample | | | | |
|---|---|---|---|---|---|
| | 7.5 g/l | 15 g/l | Control | 30 g/l | 50 g/l |
| | [g] | [g] | [g] | [g] | [g] |
| Orange juice concentrate (60° Brix) | 104.9 | 104.9 | 104.9 | 99.9 | 96.6 |
| Pineapple juice concentrate (60° Brix) | 42.0 | 42.0 | 42.0 | 37.0 | 33.7 |
| Banana powder (50%) | 60.0 | 60.0 | 60.0 | 55.0 | 51.7 |
| Mango Puree (16-20° Brix) | 20.0 | 20.0 | 20.0 | 15.0 | 11.7 |
| Ascorbic acid | 0.5 | 0.5 | 0.5 | 0.5 | 0.5 |
| Sodium benzoate | 0.1 | 0.1 | 0.1 | 0.1 | 0.1 |
| potassium sorbate | 0.1 | 0.1 | 0.1 | 0.1 | 0.1 |
| high methoxy pectin (2% solution) | 100.0 | 100.0 | 100.0 | 95.0 | 91.7 |
| Rosphips powder 200 μm | 7.5 | 15.0 | — | 30.0 | 50.0 |
| Apocarotenal 10% WS/N | — | — | 0.01 | — | — |
| Water | 665.3 | 657.0 | 672.3 | 667.3 | 664.0 |
| total | 1000 | 1000 | 1000 | 1000 | 1000 |
| Rosehip powder/100 ml serving | 0.75 | 1.5 | | 3 | 5 |

### Preparation

Sodium benzoate and potassium sorbate were dissolved into an aliquot of the water while stirring. The ascorbic acid, pectin solution and colour were dissolved and added. Then the rosehip powder was dispersed. All the juice concentrates were added and mixed well and then allowed to rest for some minutes. Next it was homogenized in a high pressure homogenizer (1 min, 150 bar/100 bar); pasteurized at 80° C. for 1 minute; and filled into translucent glass bottles and stored at a cool temperature. Serving size=100 ml

Rose hips changed the color and viscosity of the fruit smoothie significantly. It also changed the sensory profiles of the smoothies and resulted in an increased off-taste from a level of 5 g/100 ml and above and a sandy mouth feeling from a level of 0.7 g/100 ml and above. The higher the level of rosehips, the lower the fruit flavour and sourness.

The milled powder was not water soluble. Thus the powder particles precipitated to the bottom of the bottle, but this is acceptable in a smoothie. The change in profile was recognised as pleasant up to ˜3 g/100 ml, but the viscosity increases at higher levels and the drink becomes difficult to drink. Homogenization of the 200 um with 5 g/100 ml was possible but difficult.

TABLE 7
Sensory of rosehip powder in a fruit smoothie (processed with rosehips powder)

| | | L.S.D. | | L.S.D. | | L.S.D. | | L.S.D. | Off- | L.S.D. |
|---|---|---|---|---|---|---|---|---|---|---|
| | sweet | 5% | sour | 5% | fruity | 5% | grainy | 5% | taste | 5% |
| Control | 3.10 | — | 3.20 | — | 3.70 | — | 1.40 | B | 1.30 | — |
| 0.7 g/100 ml | 3.10 | — | 3.40 | — | 3.70 | — | 2.30 | A | 1.40 | — |
| 1.5 g/100 ml | 3.10 | — | 2.90 | — | 3.80 | — | 2.70 | A | 1.50 | — |
| ANOVA | not sign. | | not sign. | | not sign. | | sign. | | not sign. | |
| L.S.D. diff. | | — | | — | | — | | 0.90 | | — |

The fruit smoothies from the above test were stored in glass bottles for four weeks at 4° C., and then sampled again. Results are given in Table 8.

TABLE 8
Sensory of rosehip powder in a fruit smoothie (processed with rosehips powder) - 4 weeks

| | | L.S.D. | | L.S.D. | | L.S.D. | | L.S.D. | Off- | L.S.D. |
|---|---|---|---|---|---|---|---|---|---|---|
| | sweet | 5% | sour | 5% | fruity | 5% | grainy | 5% | taste | 5% |
| Control | 3.83 | — | 3.50 | — | 4.17 | — | 1.17 | B | 1.25 | — |
| 3 g/100 ml | 3.75 | — | 3.50 | — | 4.08 | — | 2.67 | A | 2.17 | — |
| 5 g/100 ml | 3.50 | — | 3.50 | — | 3.92 | — | 3.17 | A | 2.33 | — |
| ANOVA | not sign. | | not sign. | | not sign. | | sign. | | not sign. | |
| L.S.D. diff. | | — | | — | | — | | 0.61 | | — |

### Example 7

### Orange Juice

Rosehip powder has been added to an orange juice with 90% juice content before production to test the possibility of high pressure homogenization and sensory acceptability of different levels. Neither the 700 μm not the 400 μm size powders could be used as neither was able to be high pressure homogenized (too coarse). With the 200 μm material we were able to produce an orange juice at with up to of 3.6 g rosehip powder per serving of 240 ml.

Rosehip powder at a level of 3.6 g/240 ml orange juice changed the sensory profile regarding graininess. The panelists detected an off-taste, mostly described as earthy. However the orange juice was still judged acceptable.

TABLE 9
Sensory of rosehip powder in an orange juice (processed with rosehips powder) 1

| | | L.S.D. | | L.S.D. | | L.S.D. | | L.S.D. | Off- | L.S.D. |
|---|---|---|---|---|---|---|---|---|---|---|
| | sweet | 5% | sour | 5% | fruity | 5% | grainy | 5% | taste | 5% |
| Control | 3.09 | — | 3.55 | — | 3.18 | — | 1.09 | B | 1.36 | — |
| 3.6 g/240 ml | 3.00 | — | 3.55 | — | 2.91 | — | 2.00 | A | 2.00 | — |
| ANOVA | not sign. | | not sign. | | not sign. | | sign. | | not sign. | |
| L.S.D. diff. | | — | | — | | — | | 0.87 | | — |

### Example 6

### Cereal Bars

Finely ground rose hip powder was incorporated into cereal bars in a variety of ways—simply mixed in during production, in a filling, or as a topping.

### Cereal Bar, Non Baked

| Ingredients | (in g) |
|---|---|
| Glucose Syrup | 184.30 |
| Sucrose | 98.00 |
| Glycerine | 25.00 |
| Water | 17.00 |
| Biscofin N (alt: Biscuitine N) | 42.00 |
| Soy Lecithin | 8.00 |
| NZMP Dairy Protein Crisp 6001 | 416.00 |
| Deyhdrated fruit pieces | 38.00 |
| Powdered strawberry flavour | 2.50 |
| Citric acid, water free | 2.50 |
| Rosehip Powder | 166.7 |
| Total | 1000.00 |

Preparation: Glucose syrup, sucrose, water and glycerine were placed into a vessel and heated to 100° C. The Biscofin fat was melted and the Soy Lecithin was added. Next, the Biscofin Fat and Soy Lecithin combination was added to glucose syrup mixture, and mixed to combine. The dry ingredients were mixed in a Kenwood mixer for about 1 minute. The glucose syrup mixture was cooled to under 50° C., then added and mixed to combine in a Kenwood mixer for about 2 minutes at a high level. The mixture was rolled on to a marble plate between baking paper, and allowed to set. Once set mixture was cut to a bar size of about 30 g

Cereal Bar, Non Baked—with Filling

### 1. Milk Cream

| Ingredients | (in g) |
|---|---|
| Lamequick ® (emulsifier) | 20.0 |
| Fine crystal sugar | 8.5 |
| Sorbit powder | 10.0 |
| Glycerine, liquid | 10.0 |
| Milk Powder, 20% fat | 25.0 |
| Butter, liquid | 35.0 |
| Coconut oil/palmkernel fat | 150.0 |
| Soy lecithin | 5.0 |
| Fructose | 35.0 |
| Powdered strawberry flavour | 1.5 |
| Rosehip powder | 200.0 |
| TOTAL | 500.0 |

Preparation: The dry ingredients were mixed in a Kenwood mixer for about 1 Minute. The melted fat and the glycerine were added to the dry mixture and mixed and combined in a Kenwood mixer for about 2 minutes at a high level.

12.5 g of filling was placed between two plates of bar from above and cut into servings of 30 g

Protein Bar with White Chocolate Topping

### 1. Chocolate Topping*

| Ingredients | (in g) |
|---|---|
| White chocolate, couverture | 500.0 |
| Coconut fat/palmkernel fat | 290.0 |
| Rosehip powder | 210.0 |
| Total | 1000.0 |

Preparation: Melted chocolate (about 45 C.°) and the melted fat were mixed and stirred to combine. It was then tempered on a marble plate to about 30.5° C. Rosehip powder was added and stirred to combine.

This recipe of a milk cream with rosehips was too sticky onto the cereal bars and melted when taken into hands; a level of approximately 1.5 g rosehip powder was judged as suitable (150 g rosehip powder/kg chocolate couverture).

Protein Bar*

| Ingredients | (in g) |
|---|---|
| Glycerine | 195.0 |
| Sucrose | 275.0 |
| Water | 51.0 |
| NZMP WPI Whey Protein Isolate | 97.0 |
| 895 | |
| Hydrogenated Coconut Oil | 108.0 |
| NZMP Dairy Protein Crisp 6001 | 108.0 |
| NZMP 4800 Dairy Protein Milk | 97.0 |
| Protein | |
| Karaya (Hydrokolloid) | 5.0 |
| Deyhdrated fruit pieces | 56.0 |
| Powdered strawberry flavour | 3.0 |
| Citric acid, water free | 5.0 |
| total approx | 1000.00 |

Preparation: Glycerine was placed into a vessel and heated to 100° C. Sucrose and Water were placed into a vessel and heated to 115° C. The WPI 895 was added to glycerine in a Kenwood Mixer and mixed to combine. The sucrose mixture was added immediately afterwards, and mixed to combine in a Kenwood mixer and stirred about 3 minutes at a high level. Next, the melted fat was added and mixed to combine and stirred about 2 minutes at a normal level. The remaining ingredients were added and mixed on low until combined. The mixture was rolled on to a marble plate between baking paper for the topping. After the cooling time of about 1 hour, the mixture was cut to a bar size. 10 g chocolate coating (above) was placed onto the cereal bar

Sensory profiles compared to the original cereal bar changes. Panellists recognized a rose hip taste and an orange color. These changes were not seen as negative. The combination of rose hip and white chocolate used as a base for the topping was judged pleasant.

What is claimed is:

1. A rosehip powder having substantially no particles larger than 600 microns, and in which 50% of the particles (by volume) have a size of 125 microns (plus or minus 25 microns), when measured by measured by laser diffraction.

2. A rosehip powder according to claim 1 which has substantially no particles larger than 500 microns.

3. A rosehip powder which has particle sizes such that 80% would pass through a sieve which has openings of 300-424 microns and at least 60% by weight would pass through a sieve which has openings of 150-200 microns.

4. A foodstuff comprising a rosehip powder having substantially no particles larger than 600 microns when measured by laser diffraction, with the proviso that the foodstuff is not tea.

5. A foodstuff according to claim 4 which is selected from the group consisting of:
a) beverages
b) cookies, cereal bars, cakes
c) yoghurt;
d) candies and confectionary, including chocolate and
e) animal foods.

6. A method of preparing a liquid food product comprising adding fine rosehip power to a liquid food to obtain a rosehip powder containing food, and homogenizing the rosehip powder containing liquid food under high pressure.

7. A nutraceutical comprising a rosehip powder having substantially no particles larger than 600 microns when measured by laser diffraction.

8. A nutraceutical according to claim 7 which is a tablet or capsule.
